(19) United States
(12) Patent Application Publication
(10) Pub. No.: US 2008/0071119 A1
(43) Pub. Date: Mar. 20, 2008

(54) Process for preparing alkoxy-pure alkaline-earth alkoxides

(75) Inventors: Burkhard Standke, Lorrach (DE); Hartwig Rauleder, Rheinfelden (DE)

Correspondence Address: VENABLE LLP, P.O. BOX 34385, WASHINGTON, DC, 20043-9998, US

(73) Assignee: Degussa AG, Dusseldorf (DE)

(21) Appl. No.: 11/980,380

(22) Filed: Oct. 31, 2007

Related U.S. Application Data
(63) Continuation of application No. 11/008,574, filed on Dec. 10, 2004.

(30) Foreign Application Priority Data
Dec. 13, 2003 (DE) 103 58 412.9

(51) Int. Cl. C07C 29/68

(52) U.S. Cl. 568/851; 568/700

(57) ABSTRACT

The present invention relates to a process for preparing a metal-free, alkoxide-pure alkaline-earth alkoxide of the general formula I
M(OR¹)₂ (I)
in which M stands for an element pertaining to the second Main Group of the Periodic Table of the Elements and R¹ represents a linear, branched or cyclic alkyl group with 2 to 20 C atoms, by alcoholysis of a compound of the general formula II
M(OR²)ₓ(OR³)_{y}(OR⁴)_{z} (II)
in which M stands for an element pertaining to the second Main Group of the Periodic Table of the Elements, groups R², R³ and R⁴ are the same or different and represent a linear alkyl group with 1 to 4 C atoms, with the proviso 0≦x≦2, 0≦y≦2, 0≦z≦2 with (x+y+z)=2, with an alcohol, which is employed in excess, of the general formula III
HOR¹ (III)
in which R¹ has the same significance as in formula I, is different from groups R², R³ and R⁴ according to formula II, and in the alkyl chain possesses at least one C atom more than the longest alkyl group from the series R², R³ and R⁴, the compounds of the formula I and/or of the formula II being kept dissolved, at least in a proportion, during the conversion.

The invention relates to a process for preparing alkaline-earth alkoxides by alcoholysis. The process is based on the exchange of alkoxide groups of a metal alkoxide in the presence of an alcohol other than the alcohol corresponding to the original alkoxide, i.e. on the alcoholysis of a metal alkoxide with a different alcohol.

Alkaline-earth dialkoxides, hereinafter also called alkaline-earth alkoxides for short, are employed in a variety of ways in organic synthetic chemistry.

The most customary process for their preparation is the direct reaction of alkaline-earth metal with alcohol subject to dehydrogenation [Liebigs Annalen der Chemie 444, 236 (1925)]. In the case where use is made of longer-chain alcohols this reaction is rendered unusually difficult by reason of the low affinity of the coreactants for one another.

U.S. Pat. No. 2,965,663 teaches the reaction of metals pertaining to Groups IA, IIA and IIIA of the Periodic Table of the Elements (PTE) with alcohols to give corresponding alkoxides by a special reflux process. The unusually long reaction-times are disadvantageous, particularly when metals pertaining to Groups IIA and IIIA are employed.

DE-OS 22 61 386 discloses that the reaction of alkaline-earth metal and alcohol can be carried out more quickly at higher temperatures, but with the disadvantage that the reaction has to be carried out in an autoclave under high pressure.

A general problem in the preparation of alkaline-earth alkoxides from alkaline-earth metal and alcohol is the residual content of unreacted metal, which is troublesome in connection with further use of the product, for example if the magnesium alkoxide is employed as a catalyst in organic synthetic chemistry.

Another route for the preparation of metal alkoxides, particularly those of higher alcohols, is alcoholysis (Kirk-Othmer Encyclopedia of Chemical Technology, 3^{rd} Ed., Vol. 2, pages 8 and 9). A disadvantageous aspect of this process is the fact that in the course of the preparation of alkoxides pertaining to Group IIA of the PTE a contamination with alcohol or alkoxide of the lower alcohol is to be observed remaining in the product. For instance, in the course of the alcoholysis of magnesium ethanolate with isopropanol to give magnesium isopropylate about 15 wt. % ethanolate, reckoned as ethanol, remains in the product. Such a content of foreign alkoxide in the product may also have a disturbing effect, for example, in connection with the use of magnesium diisopropanolate as a catalyst in the course of a synthesis in organic chemistry.

The object underlying the present invention was consequently to make available a further process, which is as economic as possible, for preparing higher alkaline-earth alkoxides having sufficient product purity.

The object is achieved, according to the invention, in accordance with the features of the Claims.

Surprisingly, it has been found that a metal-free and alkoxide-pure alkaline-earth alkoxide of the general formula I
M(OR¹)₂ (I)

in which M stands for an element pertaining to the second Main Group of the Periodic Table of the Elements and R¹ represents a linear, branched or cyclic alkyl group with 2 to 20 C atoms, preferably with 2 to 10 C atoms, can be prepared in straightforward and, at the same time, economic manner if a compound in solution of the general formula II
M(OR²)ₓ(OR³)_{y}(OR⁴)_{z} (II)

in which M stands for an element pertaining to the second Main Group of the Periodic Table of the Elements, preferably for Mg, Ca, groups R², R³ and R⁴ are the same or different and represent a linear alkyl group with 1 to 4 C atoms, with the proviso 0≦x≦2, 0≦y≦2, 0≦z≦2 with (x+y+z)=2, is converted with an alcohol, which is employed in excess, of the general formula III
HOR¹ (III)

in which R¹ has the same significance as in formula I, is different from groups R², R³ and R⁴ according to formula II, and in the alkyl chain possesses at least one C atom more than the longest alkyl group from the series R², R³ and R⁴, the compounds of the formula I and/or of the formula II being kept dissolved, at least in a proportion, during the alcoholysis.

According to the invention, in the course of the present process care has to be taken in particular to ensure that for the purpose of carrying out the alcoholysis such a starting alkoxide (compare compounds of the formula II) is employed and/or in the course of the present conversion a reaction intermediate arises, for example a mixed alkoxide, and/or a target alkoxide arises—that is to say, a product of the formula I—that is soluble, at least in a proportion, in the alcohol of the formula III that is used for the conversion.

Furthermore, the reaction mixture for carrying out the alcoholysis is expediently heated. The present conversion is completed, as a rule, by the removal by distillation of the alcohol that is liberated in the course of the alcoholysis:
M(OR² )ₓ(OR³)_{y}(OR⁴)_{z}+2 R¹OH→M(OR¹)₂+x HOR²+y HOR³+z HOR⁴

The alcoholysis of magnesium dimethanolate dissolved in methanol with n-hexanol may be cited in exemplary manner:
Mg(OMe)₂+2 HexOH→Mg(OHex)₂+2 MeOH

The conversion of pulverulent calcium diethanolate in n-octanol may be given as a further example:
Ca(OEt)₂+2 OctOH→Ca(O-Oct)₂+2 EtOH

Moreover,
Mg (O-n-propyl)₂, Ca (O-n-propyl)₂
Mg(O-i-propyl)₂, Ca(O-i-propyl)₂
Mg (O-n-butyl)₂, Mg(O-n-butyl)₂
Mg(O-i-butyl)₂, Mg(O-i-butyl)₂

in particular are accessible by the process according to the invention.

In connection with the present invention the expression ‘metal-free alkaline-earth alkoxides’ is to be understood to mean those which contain less than 0.04 wt. % alkaline-earth metal, relative to the alkaline-earth alkoxide. In the case of the present invention, preferentially metal-free alkaline-earth alkoxides of the general formula I with ≦0.03 wt. % alkaline-earth metal, in particular those with ≦0.02 wt. % alkaline-earth metal right down to the detection limit of the alkaline-earth metal in question, are obtained, the stated figure being relative to the alkaline-earth alkoxide in each case.

In connection with the present invention the expression ‘alkoxide-pure alkaline-earth alkoxides’ is to be understood to mean those which contain ≦10 wt. % foreign alkoxide, reckoned as alcohol and relative to the desired alkaline-earth alkoxide. In the case of the present invention, preferentially alkoxide-pure alkaline-earth alkoxides of the general formula I with ≦5.0 wt. % foreign alkoxide, particularly preferably those with ≦1.0 wt. % foreign alkoxide right down to the detection limit of the foreign alkoxide or alcohol in question, are obtained under the given conditions, the stated figure being relative to the alkaline-earth alkoxide in each case.

The present invention therefore provides a process for preparing a metal-free and alkoxide-pure alkaline-earth alkoxide of the general formula I
M(OR¹)₂ (I),

in which M stands for an element pertaining to the second Main Group of the Periodic Table of the Elements and R¹ represents a linear, branched or cyclic alkyl group with 2 to 20 C atoms,

by alcoholysis of a compound of the general formula II
M(OR²)ₓ(OR³)_{y}(OR⁴)_{z} (II),

in which M stands for an element pertaining to the second Main Group of the Periodic Table of the Elements, groups R², R³ and R⁴ are the same or different and represent an alkyl group with 1 to 4 C atoms, with the proviso 0≦x≦2, 0≦y≦2, 0≦z≦2 with (x+y+z)=2,

with an alcohol, which is employed in excess, of the general formula III
HOR¹ (III)

in which R¹ has the same significance as in formula I, is different from groups R², R³ and R⁴ according to formula II, and in the alkyl chain possesses at least one C atom more than the longest alkyl group from the series R², R³ and R⁴,

the compounds of the formula I and/or of the formula II being kept dissolved, at least in a proportion, during the conversion.

Magnesium dimethanolate or magnesium diethanolate or calcium dimethanolate is preferably employed by way of compound of the general formula II in the process according to the invention.

Moreover, ethanol, n-propanol, i-propanol, n-butanol, sec-butanol, t-butanol, n-pentanol, amyl alcohol, n-hexanol, n-octanol, i-octanol or n-decanol is preferentially employed by way of compound of the general formula III in the process according to the invention.

With a view to carrying out the process according to the invention, an alcohol of the general formula III is preferably charged, and an alkaline-earth metal alkoxide of the general formula II is added, subject to good intermixing. The alkaline-earth alkoxide may expediently be added in powder form or in dispersed form or in dissolved form. In particular, it is preferred that the alkaline-earth alkoxide is added dissolved in methanol and/or ethanol or dispersed in methanol and/or ethanol. Homogeneous solutions of the alkaline-earth alkoxide are preferentially employed.

In order to be able to carry out the alcoholysis according to the invention particularly quickly, working may proceed at a temperature higher than the ambient temperature. The conversion according to the invention is preferably carried out at a temperature within the range from 20°0 C. right up to the boiling-points under normal pressure of the alcohols that are present in the given case. In particular, working proceeds at a temperature within the range from 90° C. to 140° C. In the course of carrying out the alcoholysis according to the invention the alcohol HOR², HOR³ and/or HOR⁴ arising during the conversion is expediently removed from the reaction mixture by distillation; a vacuum may also be applied for this purpose. In the course of carrying out the present process, care furthermore has to be taken to ensure that the distillation system that is used has an adequate separation efficiency. With a view to removing the stated alcohol from the reaction mixture by distillation, working preferably proceeds at a pressure of less than 0.1 bar to 1.1 bar abs.

The conversion according to the invention is preferentially carried out under normal pressure, or optionally under slightly reduced pressure, until such time as the corresponding boiling-temperature of the alcohol having the highest boiling-point can be detected at the head of the column for at least one hour.

Magnesium methanolate, prepared expediently by dissolving magnesium in methanol, is generally soluble in methanol with a concentration of up to 10 wt. % and, in connection with the process according to the invention, is therefore preferred as educt, i.e. as starting alcoholate of the general formula II. Likewise, for an advantageous progress of the conversion according to the invention calcium ethanolate, for example, is preferred, in which case the preparation thereof may be undertaken by dissolving calcium in ethanol.

In particular, alkoxides of the formula II may be converted in accordance with the invention with higher alcohols such as, for example, n-hexanol or n-octanol.

In general, the process according to the invention is carried out as follows:

Firstly an alkaline-earth alkoxide of the general formula II M(OR²)ₓ(OR³)_{y}(OR⁴)_{z} is prepared in a manner known per se. To this end, an alkaline-earth metal M may be caused to react in an alcohol or alcohol mixture, consisting of HOR², HOR³ and/or HOR⁴, corresponding to the respective alkoxide, preferably methanol or ethanol. The surface of the metal that is employed may additionally be precleaned, in order to obtain a better kick-off of the reaction. Furthermore, a catalyst, iodine for example, may be added. If the excess alcohol is separated off after the reaction, a pulverulent metal alkoxide may be obtained. Ordinarily an alkaline-earth alkoxide prepared in this way contains a residual portion of alkaline-earth metal amounting to ≧0.04 wt. %, relative to the alkaline-earth alkoxide, particularly when use is made of alcohols with more than 2 C atoms. As a rule, the alkaline-earth alkoxide is handled subject to exclusion of moisture and under protective-gas atmosphere. The alkaline-earth alkoxide—that is to say, the educt—can now be employed in the form of an alcoholic solution or in powder form or in dispersed form, for example as an alcoholic dispersion, for the alcoholysis according to the invention. The alcohol provided for the alcoholysis, which is expediently dried and which is of the general formula III (HOR¹), is expediently charged in excess in a dry, coolable or heatable reaction vessel with stirring device under protective gas, for example dry nitrogen or argon, and the educt according to formula II M(OR²)ₓ(OR³)_{y}(OR⁴)_{z}—dispersed, partially dissolved or dissolved in HOR¹, HOR², HOR³ and/or HOR⁴—is added, and the educt reacts in accordance with the invention with the alcohol HOR¹, preferably subject to formation of HOR², HOR³ as well as HOR⁴ and M(OR¹)₂, whereby the reaction mixture is expediently well intermixed, the temperature is preferably maintained within the range from 20° C. to 140° C., preferentially 90-140° C., and during the alcoholysis according to the invention HOR², HOR³, HOR⁴ or HOR¹, or appropriate mixtures thereof, are simultaneously removed from the system via the gas phase—that is to say, by distillation. As a rule, the desired product M(OR¹)₂ is obtained in this way, dissolved, partially dissolved or dispersed in the alcohol (HOR¹) corresponding to the target alkoxide. Now the residual alcohol or the residual alcohol mixture can be separated by distillation or by filtration from the alkaline-earth alkoxide M(OR¹)₂ obtained.

As a rule, in advantageous manner less than 10 hours are required for the implementation, in accordance with the invention, of one batch.

Metal-free and alkoxy-pure alkaline-earth alkoxides of higher alcohols are accessible in straightforward and economic manner—also on a technical scale—by the process according to the invention.

The present invention will be elucidated in more detail by the following Examples, without restricting the subject-matter of the invention:

### EXAMPLES

In the following Examples 1 to 7 the educt alkoxide is present in homogeneous solution. After alcoholysis according to the invention, products are obtained having a foreign-alkoxide content of ≦1 wt. %, reckoned as alcohol.

In Examples 8 and 9 both the product alkoxide and the educt alkoxide are present in homogeneous solution. After alcoholysis, a product is obtained having a foreign-alkoxide content of ≦0.1 wt. %, reckoned as alcohol.

If the initial alkaline-earth alkoxides are sparingly soluble in the solvent that is used—that is to say, in the respective alcohol or alcohol mixture—see Comparative Example A, then the alcoholysis to the target alkoxide is undertaken only incompletely, so that a foreign-alkoxide content >10 wt. % remains in the product.

### Example 1

Preparation of magnesium diisopropanolate from magnesium dimethanolate

In an apparatus—consisting of 2 l multinecked flask with internal thermometer, KPG calibrated precision-glass stirrer, dropping funnel, distillation column (packed column, inside diameter 25 mm, filling level 1.2 m, wire-gauze rings 4*4 mm V4A), with column head (automatically controlled liquid distributor, contact thermometer), heating mantle and N₂ blanketing—1.2 kg isopropanol is charged and heated to boiling. At a rate of feed of about 15 ml/minute, methanolic magnesium-dimethanolate solution (9.5 wt. % magnesium methylate) is metered in. At the start of the metering, with a reflux ratio of 1 and with an overhead temperature falling from 82° C. to 65° C., firstly an isopropanol/methanol mixture is distilled off, and at 65° C. pure methanol is distilled off. Subsequently, with rising overhead temperature (from 65° C. to 82° C.), distillation is effected with a reflux ratio rising from 5 to 20. Once the overhead temperature has been constant at 82° C. for a fairly long time (about 1 hour), the distillate consists of isopropanol, and the reaction is concluded. The total duration of the conversion amounts to about 7 hours. Magnesium diisopropanolate is sparingly soluble in propanol. At the end of the reaction it is present dispersed in propanol. The dispersion is concentrated by evaporation on a Rotavapor rotary evaporator and dried for 3 hours at about 120° C. and at a pressure of less than 1 mbar. A white, fine-grained powder (primary particles about 1 μm in diameter, agglomerated into particles measuring 5 μm to 50 μm) is obtained. The methanol content of the isolated magnesium isopropanolate amounts to less than 1 wt. %. At less than 0.02 wt. % the content of free magnesium lies below the detection limit of the chosen analytical method.

### Example 2

Preparation of magnesium di-n-butanolate from magnesium dimethanolate

In an apparatus—consisting of 2 l multinecked flask with internal thermometer, KPG calibrated precision-glass stirrer, dropping funnel, distillation column (packed column, inside diameter 25 mm, filling level 1.2 m, wire-gauze rings 4*4 mm V4A), with column head (automatically controlled liquid distributor, contact thermometer), heating mantle and N₂ blanketing—1.2 kg n-butanol (butane-1-ol) is charged and heated to 95° C. At a rate of feed of about 15 ml/minute, 0.8 kg methanolic magnesium-dimethanolate solution (7.5 wt. % magnesium methylate) is metered in. At a bubble temperature rising from 90° C. to 117° C., firstly pure methanol is distilled off at an overhead temperature of 65° C. Subsequently, with rising overhead temperature (from 65° C. to 117° C.) with a reflux ratio of 20, a methanol/n-butanol mixture is distilled off. Once the overhead temperature has been constant at 117° C. for a fairly long time (about 1 hour), the distillate consists of n-butanol, and the reaction is concluded. The reaction-time amounts to about 7 hours. Magnesium di-n-butanolate is sparingly soluble in n-butanol. At the end of the reaction it is present dispersed in n-butanol. The dispersion is concentrated by evaporation on a Rotavapor rotary evaporator and is dried for 3 hours at about 120° C. and at a pressure of less than 1 mbar. A white, coarse-grained powder, crushed in gel-like manner with irregular particle structure, is obtained. The methanol content in the isolated magnesium di-n-butanolate amounts to less than 0.11 wt. %. At less than 0.02 wt.-% the content of free magnesium lies below the detection limit of the chosen analytical method.

### Example 3

Preparation of magnesium di-sec-butanolate from magnesium dimethanolate

In an apparatus—consisting of 2 l multinecked flask with internal thermometer, KPG calibrated precision-glass stirrer, dropping funnel, distillation column (packed column, inside diameter 25 mm, filling level 1.2 m, wire-gauze rings 4*4 mm V4A), with column head (automatically controlled liquid distributor, contact thermometer), heating mantle and N₂ blanketing—0.8 kg sec-butanol (butane-2-ol) is charged and heated to 95° C. At a rate of feed of about 15 ml/minute, 0.2 kg methanolic magnesium-dimethanolate solution (7.5 wt. % magnesium methylate) is metered in. At a bubble temperature rising from 90° C. to 99° C., firstly pure methanol is distilled off at an overhead temperature of 65° C. Subsequently, with a rising overhead temperature (from 65° C. to 99° C.) with a reflux ratio of 20, a methanol/butanol mixture is distilled off. Once the overhead temperature has been constant at 99° C. for a fairly long time (about 1 hour), the distillate consists of sec-butanol, and the reaction is concluded. The reaction-time amounts to about 7 hours. Magnesium di-sec-butanolate is sparingly soluble in sec-butanol. At the end of the reaction it is present dispersed in sec-butanol. The dispersion is concentrated by evaporation on a Rotavapor rotary evaporator and dried for 3 hours at about 120° C. and at a pressure of less than 1 mbar. A white, coarse-grained powder, crushed in gel-like manner with irregular particle structure, is obtained. The methanol content of the isolated magnesium sec-butylate amounts to less than 1.0 wt. %. At less than 0.02 wt.-% the content of free magnesium lies below the detection limit of the chosen analytical method.

### Example 4

Preparation of magnesium di-n-amylate from magnesium dimethanolate

In a vacuum apparatus—consisting of 2 l multinecked flask with internal thermometer, KPG calibrated precision-glass stirrer, dropping funnel, distillation column (packed column, inside diameter 25 mm, filling level 1.2 m, wire-gauze rings 4*4 mm V4A), with column head (automatically controlled liquid distributor, contact thermometer), heating mantle and N₂ blanketing, LEYBOLD (D2A) vacuum pump and cold trap (−78° C.)—1.2 kg n-amyl alcohol is charged and heated to 90° C. at 600 mbar. At a rate of feed of about 15 ml/minute, 0.8 kg methanolic magnesium-dimethanolate solution (7.5 wt. % magnesium methylate) is metered in. At a bubble temperature rising from 90° C. to 120° C., firstly pure methanol is distilled off at an overhead temperature of 54° C. Subsequently, at 400 mbar and at an overhead temperature rising as far as 104° C. with a reflux ratio of 10, a methanol/n-amyl-alcohol mixture is distilled off. Once the overhead temperature has been constant at 104° C. for a fairly long time (about 1 hour), the distillate consists of n-amyl alcohol, and the reaction is concluded. The reaction-time amounts to about 5 hours. Magnesium di-n-amylate is sparingly soluble in n-amyl alcohol. At the end of the reaction it is present dispersed in n-amyl alcohol. The dispersion is concentrated by evaporation on a Rotavapor rotary evaporator and dried for 3 hours at about 120° C. and at a pressure of less than 1 mbar. A white, coarse-grained, powder, crushed in gel-like manner with irregular particle structure, is obtained. The methanol content of the isolated magnesium di-n-amylate amounts to less than 1.0 wt. %. At less than 0.02 wt.-% the content of free magnesium lies below the detection limit of the chosen analytical method.

### Example 5

Preparation of magnesium di-n-hexanolate from magnesium dimethanolate

In a vacuum apparatus—consisting of 2 l multinecked flask with internal thermometer, KPG calibrated precision-glass stirrer, dropping funnel, distillation column (packed column, inside diameter 25 mm, filling level 1.2 m, wire-gauze rings 4*4 mm V4A), with column head (automatically controlled liquid distributor, contact thermometer), heating mantle and N₂ blanketing, LEYBOLD (D2A) vacuum pump and cold trap (−78° C.)—0.8 kg n-hexyl alcohol (hexane-1-ol) is charged and heated to 100° C. at 500 mbar. At a rate of feed of about 15 ml/minute, 0.6 kg methanolic magnesium-dimethanolate solution (7.5 wt. % magnesium methylate) is metered in. At a bubble temperature rising from 100° C. to 120° C., firstly pure methanol is distilled off at an overhead temperature of 46° C. Subsequently further distillation is effected at 350 mbar and at an overhead temperature falling to room temperature. Once the overhead temperature has fallen to room temperature, the reaction is concluded. The reaction-time amounts to about 4 hours. Magnesium di-n-hexanolate is sparingly soluble in n-hexyl alcohol. At the end of the reaction it is present dispersed in n-hexyl alcohol. The dispersion is concentrated by evaporation on a Rotavapor rotary evaporator and dried for 3 hours at about 120° C. and at a pressure of less than 1 mbar. A white, coarse-grained powder, crushed in gel-like manner with irregular particle structure, is obtained. The methanol content of the isolated magnesium n-hexylate amounts to less than 0.1 wt. %. At less than 0.02 wt.-% the content of free magnesium lies below the detection limit of the chosen analytical method.

### Example 6

Preparation of magnesium di-n-decanolate from magnesium dimethanolate

In an apparatus—consisting of 1 l multinecked flask with internal thermometer, KPG calibrated precision-glass stirrer, dropping funnel, distillation column (packed column, inside diameter 25 mm, filling level 1.2 m, wire-gauze rings 4*4 mm V4A), with column head (automatically controlled liquid distributor, contact thermometer), heating mantle and N₂ blanketing—0.25 kg n-decyl alcohol (decane-1-ol) is charged and heated to 120° C. At a rate of feed of about 25 ml/minute, 0.25 kg methanolic magnesium-dimethanolate solution (7.5 wt. % magnesium methylate) is metered in. Methanol is distilled off at an overhead temperature of 65° C., falling to room temperature. Once the overhead temperature has fallen to room temperature, the reaction is concluded. The reaction-time amounts to about 3 hours. Magnesium di-n-decanolate is sparingly soluble in n-decyl alcohol. At the end of the reaction it is present dispersed in n-decyl alcohol. The dispersion is concentrated by evaporation on a Rotavapor rotary evaporator and dried for 3 hours at about 130° C. and at a pressure of less than 1 mbar. A white, coarse-grained powder, crushed in gel-like manner with irregular particle structure, is obtained. The methanol content of the isolated magnesium di-n-decanolate amounts to less than 0.1 wt. %. At less than 0.02 wt.-% the content of free magnesium lies below the detection limit of the chosen analytical method.

### Example 7

Preparation of magnesium diethanolate from magnesium dimethanolate

In an apparatus—consisting of 2 l multinecked flask with internal thermometer, KPG calibrated precision-glass stirrer, dropping funnel, distillation column (packed column, inside diameter 25 mm, filling level 1.2 m, wire-gauze rings 4*4 mm V4A), with column head (automatically controlled liquid distributor, contact thermometer), heating mantle and N₂ blanketing—0.711 kg ethanol is charged and heated to boiling. At a rate of feed of about 1.75 ml/minute, 700 ml methanolic magnesium-dimethanolate solution (8.5 wt. % magnesium methylate) are metered in. At the start of the metering, with a reflex ratio falling from 30 to 10 and with an overhead temperature falling from 78° C. to 65° C., firstly an ethanol/methanol mixture is distilled off, and at 65° C. pure methanol is distilled off. Subsequently an ethanol/methanol mixture is distilled with rising overhead temperature (from 65° C. to 78° C.) with a reflux ratio of 10. Once the overhead temperature has been constant at 78° C. for a fairly long time (about 1 hour), the distillate consists of ethanol, and the reaction is concluded. The reaction-time amounts to about 20 hours. Magnesium diethanolate is sparingly soluble in ethanol. At the end of the reaction it is present dispersed in ethanol. The dispersion is concentrated by evaporation on a Rotavapor rotary evaporator and dried for 3 hours at about 120° C. and at a pressure of less than 1 mbar. A white, coarse-grained powder (primary particles about 1 μm in diameter, aggregated into agglomerates measuring 5 μm to 20 μm) is obtained. The methanol content of the isolated magnesium methylate amounts to less than 0.7 wt. %. At less than 0.02 wt.-% the content of free magnesium lies below the detection limit of the chosen analytical method.

### Example 8

Preparation of calcium n-hexanolate from calcium diethanolate

In an apparatus—consisting of 2 l multinecked flask with internal thermometer, KPG calibrated precision-glass stirrer, dropping funnel, distillation column (packed column, inside diameter 25 mm, filling level 1.2 m, wire-gauze rings 4*4 mm V4A), with column head (automatically controlled liquid distributor, contact thermometer), heating mantle and N₂ blanketing—84.7 g calcium diethanolate and 1.3 1 hexane-1-ol are charged. At a bottom temperature of 135° C. the calcium methylate is completely dissolved. At a bottom temperature rising from 78° C. to 148° C. and with an overhead temperature increasing from 78° C. to 98° C., the principal amount of ethanol is removed. Subsequently the bottom temperature is increased to 158° C., the overhead temperature rising to 157° C. Once the overhead temperature has been constant for a fairly long time (about 0.5 hour), the reaction is concluded, and the overhead product consists of hexanol. The reaction-time amounts to about 3 hours. After complete removal of the hexanol on a Rotavapor rotary evaporator (3 hours, 180° C., p<1 mbar), yellow, coarse-grained, calcium n-hexanolate, crushed in gel-like manner, is isolated. The ethanol content amounts to less than 0.1%.

### Example 9

Preparation of calcium di-n-octanolate from calcium diethanolate

In an apparatus—consisting of 1 l multinecked flask with internal thermometer, KPG calibrated precision-glass stirrer, distillation column (packed column, inside diameter 25 mm, filling level 1.2 m, wire-gauze rings 4*4 mm V4A), with column head (automatically controlled liquid distributor, contact thermometer), heating mantle and N₂ blanketing—34 g calcium diethanolate and 650 ml octane-1-ol are charged. At a bottom temperature of 141° C. the calcium diethanolate is completely dissolved. At a bottom temperature rising from 78° C. to 165° C. and at an overhead temperature increasing from 78° C. to 120° C., firstly ethanol is distilled off, and subsequently an ethanol-/n-octanol mixture is distilled off. Once the overhead temperature falls to room temperature with a bottom temperature of 175° C., the reaction is concluded. The reaction-time amounts to about 3 hours. After complete removal of the octanol on a Rotavapor rotary evaporator (3 hours, 180° C., p<1 mbar), yellow, coarse-grained calcium di-n-octanolate, crushed in gel-like manner, is isolated. The ethanol content amounts to less than 0.1%.

### Comparative Example A

Preparation of magnesium isopropanolate from magnesium diethanolate

In an apparatus—consisting of 2 l multinecked flask with internal thermometer, KPG calibrated precision-glass stirrer, distillation column (packed column, inside diameter 25 mm, filling level 1.2 m, wire-gauze rings 4*4 mm V4A), with column head (automatically controlled liquid distributor, contact thermometer), heating mantle and N₂ blanketing—229 g magnesium methylate and 1.5 1 isopropanol (propane-2-ol) are charged. At a bottom temperature of 82° C., at an overhead temperature of 78° C. to 80° C., ethanol is distilled off (duration of distillation 24 hours). Subsequently isopropanol is removed in a Rotavapor rotary evaporator, and the remaining powder is dried for 2 hours in the vacuum (p<1 mbar, T=80° C.). Despite a long duration of conversion (24 hours), the isolated product still contains 15 wt. % ethanol after hydrolysis.

### Comparative Example B

Preparation of magnesium di-n-propanolate in the autoclave

In a 10 1 steel autoclave 112 g magnesium filings and 3 000 g propane-1-ol are charged. At 188° C. and at a pressure of 38 bar the conversion is carried out in a total of 5 hours. Subsequently n-propanol is removed by distillation at a temperature of 80° C. and at a pressure of about 50 mbar. The alkoxide is subsequently dried at 80° C. and at a pressure <1 mbar. The product contains metallic magnesium in a quantity of 0.04 wt. %, relative to magnesium di-n-propanolate.

1. A process for preparing a metal-free, alkoxide-pure alkaline-earth alkoxide of the general formula I
M(OR¹)₂ (I)
in which M stands for an element pertaining to the second Main Group of the Periodic Table of the Elements and R¹ represents a linear, branched or cyclic alkyl group with 2 to 20 C atoms,
by alcoholysis of a compound in solution of the general formula II
M(OR²)ₓ(OR³)_{y}(OR⁴)_{z} (II)
in which M stands for an element pertaining to the second Main Group of the Periodic Table of the Elements, groups R², R³ and R⁴ are the same or different and represent a linear alkyl group with 1 to 4 C atoms, with the proviso 0≦x≦2, 0≦y≦2, 0≦z≦2 with (x+y+z)=2,
with an alcohol, which is employed in excess, of the general formula III
HOR¹ (III)
in which R¹ has the same significance as in formula I, is different from groups R², R³ and R⁴ according to formula II, and in the alkyl chain possesses at least one C atom more than the longest alkyl group from the series R², R³ and R⁴, the compounds of the formula II being kept dissolved, at least in a proportion, during the conversion.

2. Process according to claim 1, characterised in that the alcohol HOR², HOR³ and/or HOR⁴ arising during the reaction is removed from the reaction mixture by distillation.

3. Process according to claim 1 or 2, characterised in that magnesium dimethanolate or calcium diethanolate is employed by way of compound of the general formula II.

4. Process according to one of claims 1 to 3, characterised in that ethanol, n-propanol, i-propanol, n-butanol, sec-butanol, t-butanol, n-pentanol, amyl alcohol, n-hexanol, n-octanol, i-octanol or n-decanol is employed by way of compound of the general formula III.

5. Process according to one of claims 1 to 4, characterised in that the conversion is carried out under normal pressure until such time as the boiling-point of the alcohol having the highest boiling-point can be detected at the head of the column for at least one hour.

6. Process according to one of claims 1 to 5, characterised in that an alcohol of the general formula III is charged and an alkaline-earth alkoxide of the general formula II is added, subject to good intermixing.

7. Process according to claim 6, characterised in that the alkaline-earth alkoxide is added in powder form or in dispersed form or in dissolved form.

8. Process according to claim 7, characterised in that the alkaline-earth alkoxide is added dissolved in methanol and/or ethanol or dispersed in methanol and/or ethanol.
